(19) United States
(12) Patent Application Publication
(10) Pub. No.: US 2013/0303564 A1
(43) Pub. Date: Nov. 14, 2013

(54) METHOD FOR TREATING A NEURODEGENERATIVE DISORDER

(71) Applicant: RUSH UNIVERSITY MEDICAL CENTER, Chicago, IL (US)

(72) Inventor: Kalipada Pahan, Skokie, IL (US)

(21) Appl. No.: 13/892,915

(22) Filed: May 13, 2013

Related U.S. Application Data
(60) Provisional application No. 61/646,022, filed on May 11, 2012.

(51) Int. Cl. A61K 31/4709; A61K 31/435; A61K 31/417; A61K 31/192; A61K 31/216

(52) U.S. Cl. CPC A61K 31/4709; A61K 31/192; A61K 31/216; A61K 31/417; A61K 31/435
USPC 514/290; 514/570; 514/510; 514/400; 514/539; 514/312

(57) ABSTRACT

Provided herein are methods and materials for treating neurodegenerative disorders. The methods may use inhibitors of small G-proteins, such as p21^{rac}, p21^{ras}, or the combination thereof. The small G-proteins may reside in glial cells and/or the substantia

### CROSS REFERENCE TO RELATED APPLICATION

This application claims priority to U.S. Provisional Application No. 61/646,022, filed May 11, 2012, which is incorporated herein by reference in its entirety.

### FIELD OF THE INVENTION

The present invention relates to the use of therapeutic agents to treat a neurological disorder.

### BACKGROUND

Neurodegenerative disorders are a group of devastating disorders of the central nervous system, in which progressive loss of structure and function of neurons, including neuronal death is observed. There are similarities that relate these diseases to one another on a subcellular level. For example, recent studies demonstrate that neuro-inflammation and oxidative stress are two hallmarks of various neurodegenerative disorders. Among various mediators capable of promoting neuro-degeneration are microglial-derived reactive oxygen species (ROS). Similarly, a variety of pro-inflammatory cytokines including tumor necrosis factor alpha (TNF-α), interleukin-1β IL-6, eicosanoids, and other immune neurotoxins are found in either cerebrospinal fluid (CSF) or affected brain regions in neurodegenerative disorders. Accordingly, inflammation and oxidative stress are important targets for neuronal protection in neurodegenerative disorders.

Despite intense investigations, no effective therapy is available to stop the onset or progression of many neurodegenerative disorders. With a better understanding of the molecular mechanisms underlying these disorders, it becomes possible to identify agents that may be used to treat and/or diagnose neurodegenerative disorders.

### SUMMARY OF THE INVENTION

Provided herein is a method for treating a subject having a neurodegenerative disorder. An inhibitor of small G-protein activation may be administered to a subject in need thereof. The small G-protein may be p21^{rac}, p21^{ras}, or a combination thereof. The inhibitor may be sodium phenylbutyrate (NaPB), geranylgeranyl transferase inhibitor (GGTI), farnesyl transferase inhibitor (FTI), or combinations thereof. The p21^{rac} and p21^{ras} may be microglial p21^{rac} and p21^{ras}. The p21^{rac} and p21^{ras} may be substantia p21^{rac} and p21^{ras}.

Also provided herein is a method of treating a neurodegenerative disorder in a subject, comprising administering an inhibitor of farnesyltransferase or geranylgeranyltransferase to a subject in need thereof.

The neurodegenerative disorder may be Parkinson's disease, Alzheimer's disease, Schizophrenia, myasthenia gravis, multiple sclerosis, microbial infections, head trauma and stroke, Pick's disease, dementia with Lewy bodies, Huntington disease, chromosome 13 dementias, Down's syndrome, cerebrovascular disease, Rasmussen's encephalitis, viral meningitis, NPSLE, amyotrophic lateral sclerosis, Creutzfeldt-Jacob disease, Gerstrnann-Straussler-Scheinker disease, transmissible spongiform encephalopathies, ischemic reperfusion damage (e.g. stroke), brain trauma, microbial infection, chronic fatigue syndrome, Mild Cognitive Impairment; and movement disorders (including ataxia, cerebral palsy, choreoathetosis, dystonia, Tourette's syndrome, kernicterus), tremor disorders, leukodystrophies (including adrenoleukodystrophy, metachromatic leukodystrophy, Canavan disease, Alexander disease, Pelizaeus-Merzbacher disease), neuronal ceroid lipofucsinoses, ataxia telangectasia, or Rett Syndrome.

NaPB may inhibit p21^{rac} and p21^{ras} activation. GGTI may inhibit p21^{rac} activation. FTI may inhibit p21^{ras} activation. GGTI may inhibit p21^{rac} activation by directly inhibiting geranylgeranyltransferase. FTI may inhibit p21^{ras} activation by directly inhibiting farnesyl transferase. The GGTI may be GGTI-298, GGTI-2154, GGTI-2166, GGTI-286, GGTI-2166, or GGTI-DU45. The FTI may be SCH6636, R115777, Tipifamib (6-[amino(4-chlorophenyl)(1-methyl-1H-imidazol-5-yl)methyl]-4-(3-chlorophenyl)-1-methylquinolin-2(1H)-one), or Lonafarnib (4-(2-(4-(8-Chloro-3,10-dibromo-6,11-dihydro-5H-benzo(5,6)cyclohepta(1,2-b)pyridin-11-yl)-1-piperidinyl)-2-oxoethyl)-1-piperidinecarboxamide).

The inhibitor may be administered orally or intravenously. The subject may be at risk of developing a neurological disorder or may already be diagnosed with a neurological disorder.

### BRIEF DESCRIPTION OF THE DRAWINGS

FIG. 1 shows the dose-dependent inhibition of NO production by NaPB in mouse and human glial cells. Primary mouse microglia were treated with different concentrations of NaPB for 6 h followed by stimulation with LPS under serum-free condition. After 24 h of stimulation, concentrations of nitrite were measured in supernatants (A) and the level of iNOS protein was monitored in cells by Western blot (B). Results are mean+SD of three different experiments. ^{a}p<0.001 vs control; ^{b}p<0.05 vs LPS; ^{c}p<0.001 vs LPS. After 5 h of stimulation, the expression of iNOS mRNA was monitored by semi-quantitative RT-PCR (C). Cells preinctibated with different concentration of trichostatin A (TSA) and sodium butyrate (NaBu) for 6 h were stimulated with LPS for 24 h under serum-free condition followed by monitoring the level of nitrite in supernatants (p). Results are mean+S.D. of three different experiments. ^{a}p<0.001 vs control; ^{b}p<0.05 vs LPS: ^{c,d}p<0.001 vs LPS. Primary human astroglia isolated from fetal brain tissues were treated with different concentrations of NaPB for 6 h followed by stimulation with IL-1β under serum-free condition. After 48 h of stimulation, concentrations of nitrite (E) were measured in supernatants. Human astroglia plated at 70-80% confluence in 12-well plates were co-transfected with 0.25 μg of phiNOS(7.2)Luc and 12.5 ng of pRL-TK using the Lipofectamine-Plus (Invitrogen). Twenty-four h after transfection, cells received NaPB. After 6 h of incubation, cells were stimulated with IL-1β (20 ng/ml) for 12 h. Firefly (ff-Luc) and Renilla (r-Luc) luciferase activities were obtained by analyzing the total cell extract (F). Data are mean+S.D. of three different experiments. ^{a}p<0.001 versus control; ^{b}p<0.05 versus IL-1β; ^{c}p<0.001 versus IL-1β.

FIG. 2 shows attenuation of NF-κB activity in mouse microglial cells by NaPB. (A) BV-2 microglial cells preincubated with 0.5 mM NaPB for 6 h were stimulated with 1 μg/ml LPS. At different minute of stimulation, the level of phospho-1κBα was monitored by Western blot. B) Cells preincubated with different concentrations of NaPB for 6 h were stimulated with LPS for 1 h followed by monitoring the DNA-binding activity of NF-←B by EMSA, (C) Cells plated in 12-well plates were co-transfected with 0.25 μg of PBIIX-Luc (an NF-κB-dependent reporter construct) and 12.5 ng of pRL-TK. Twenty-four h after transfection, cells received different concentrations of NaPB. After 6 h of incubation, cells were stimulated with LPS for 4 h. Firefly (ff-Luc) and Renilla (r-Luc) luciferase activities were obtained by analyzing the total cell extract. Results are mean+S.D. of three different experiments, ^{a}p<0.001 vs control; ^{b}p<0.05 vs LPS; ^{c}p<0.001 vs LPS. D) Cells preincubated with 0.5 mM NaPB for 6 h were stimulated with LPS for 2 h followed by monitoring the recruitment of RelA p65 to the mouse iNOS promoter by ChIP assay. E) Cells were co-transfected with 0.25 μg of PBIIX-Luc and 12.5 ng of pRL-TK. Twenty-four hours after transfection, cells were incubated with NaPB in the presence or absence of HMG-CoA, mevalonate, FPP, GGPP, cholesterol, and coenzyme Q. After 6 h of incubation, cells were stimulated with LPS for 4 h followed by assay of firefly (ff-Luc) and Renilia (r-Luc) luciferase activities. Results are mean+S.D. of three different experiments. ^{a}p<0.001 vs control ^{b}p<0.001 vs LPS; ^{c}_{p<}0.001 vs LPS+NaPB.

FIG. 3 shows the inhibition of the production of ROS in mouse microglial cells by NaPB. Cells were treated with 500 μM NaPB for 6 h followed by stimulation with 1 μM MPP+. At 15 min of stimulation, the generation of ROS was monitored by carboxy-H₂DCFDA (A). At different intervals (measured in minutes), superoxide production was assayed in whole cells (B). Cells preincubated with 500 μM NaPB for 6 h were stimulated with LPS (1 μg/ml), TNF-α (50 ng/ml), IL-1β (20 ng/ml), gp120 (200 pg/ml), and fibrillar Aβ1-42 (1 μM). At 10 min of stimulation, superoxide production was assayed in whole cells (C). Results are mean+SD of three different experiments. ^{a}p<0.001 vs control; ^{b}p<0.001 vs stimuli. D) Cells were incubated with NaPB in the presence or absence of HMG-CoA, mevalonate, GGPP, FPP, cholesterol, and coenzyme Q. After 6 h of incubation, cells were stimulated with LPS for 10 min followed by assay of superoxide. Results are mean+SD of three different experiments. ^{a}p<0.001 vs control; ^{b}p<0.001 vs LPS; ^{c} p<0.001 vs LPS+NaPB.

FIG. 4 shows the inhibition of the activation of NF-κB and the production of ROS by NaPB via modulation of p21^{ras} and p21^{rac}. A) Mouse BV-2 microglial cells preincubated with different concentrations of FTI and GGTI were stimulated with LPS for 60 min followed by monitoring the activation of NF-κB by EMSA. B) Cells preincubated with different concentrations of FTI and GGTI were stimulated with LPS for 10 min followed by monitoring the production of superoxide. Results are mean+SD of three different experiments. ap<0.001 vs control; bp<0.001 vs LPS. C) Cells were transfected with Δp21^{ras}, Δp21^{rac} or an empty vector using Lipofectamine Plus. Twenty-four hour after transfection, cells were stimulated with LPS followed by monitoring superoxide production at 15 and 30 ‘rain of stimulation. ^{a}p<0.001 vs control; ^{b}p<0.001 vs LPS-15 min; ^{c}p<0.001 vs LPS-30 min. D) Cells preincubated with 500 μM NaPB were stimulated with LPS. At different time points, activation of p21^{ras} and p21^{rac} was monitored. Results represent three independent experiments.

FIG. 5 shows the role of p21^{ras} and p21^{rac} in the induction of iNOS and the activation of NF-κB in microglial cells. A) Cells plated in 12-well plates were co-transfected with 0.2 μg PBIIX-Luc and 0.2 μg Δp21^{ras}, Δp21^{rac} or an empty vector. Each transfection also included 12.5 ng of pRL-TK. Twenty-four hour after transfection, cells were stimulated with LPS for 4 h. Firefly (ff-Luc) and Renilla (r-Luc) luciferase activities were obtained by analyzing the total cell extract. Results are mean+S.D. of three different experiments, ap<0.001 vs control; bp<0.001 vs LPS. B) Cells were transfected with Δp21^{ras}, Δp21^{rac} or an empty vector. Twenty-four hour after transfection, cells were stimulated with LPS for 5 h under serum-free condition followed by monitoring the expression of iNOS mRNA RT-PCR. Cells were transfected with either control siRNA or Ras siRNA and 24 h after transfection, cells were stimulated with LPS for 5 h followed by monitoring the expression of Ras protein by Western blot (C) and iNOS mRNA by RT-PCR (D). After transfection, cells were stimulated with LPS for 24 h followed by monitoring the level of nitrite in supernatants (E). ^{a}p<0.001 vs control; ^{b}p<0.001 vs LPS. F) Cells plated in plates were co-transfected with 0.2 μg PBIIX-Luc and different amounts of either RasV12 (a constitutively-active mutant of p21^{rac}) or control vector. Each transfection also included 12.5 ng of pRL-TK. Twenty-four hour after transfection, cells were treated with NaPB for 6 h under serum-free condition followed by monitoring firefly (ff-Luc) and Renilla (r-Luc) luciferase activities in total cell extract. G) Under similar condition, the effect of RasV 12 and an empty vector on the expression of iNOS mRNA was monitored in cells. Results are mean+S.D. of three different experiments. ^{a}p<0.001 vs empty vector (0.2 μg); ^{b}p<0.001 vs RasV12 (0.1 μg); ^{c}p<0.001 vs RasV12 (0.2 μg).

FIG. 6 shows the activation of small G proteins (p21^{ras} and p21^{rac}) and NF-κB in ventral midbrain of MPTP-intoxicated mice is NaPB-sensitive. A) Mice were treated with NaPB (200 mg/kg body wt/d.) via gavage from 1 d prior to MPTP injection. Six h after the last injection of MPTP, activation of p21^{ras} and p21^{rac} was monitored in ventral midbrain tissues. Experiment was repeated three times each time using two animal in each group. B) Bands from three different mice were quantified and activation of p21^{ras} and p21^{rac} is shown as percent of control, C) Mice were treated with NaPB (200 mg/kg body wt/d) from 3 h after the last injection of MPTP. Twenty-four h after the last injection of MPTP, ventral midbrain sections were immunostained for p65 (low magnification). Midbrain sections of MPTP-intoxicated mice were also double-labeled for p65 and glial cell markers (GFAP for astrocytes and CD11b for microglia). Results represent three independent experiments. D) NF-κB p65 positive cells counted in four nigral sections (two images per slide) from each of four mice in an Olympus IX81 fluorescence microscope using the MicroSuite™ imaging software are mentioned as cells/mm2. ^{a}p<0.0001 vs saline-control; ^{b}p<0.0001 vs MPTP.

FIG. 7 shows the expression of proinflammatory molecules in ventral midbrain of MPTP-intoxicated mice is NaPB-sensitive. A) Mice were treated with NaPB (200 mg/kg body wt/d) from 3 h after the last injection of MPTP. Twenty-four h after the last injection of MPTP, ventral midbrain sections were immunostained for iNOS (low magnification), Midbrain sections of MPTP-intoxicated mice were also double-labeled for iNOS and glial cell markers (GFAP for astrocytes and CD11b for microglia), Results represent three independent experiments. B) Cells positive for iNOS were counted in four nigral sections (two images per slide) from each of four mice, ap<0.0001 vs saline-control; bp<0.0001 vs MPTP. The mRNA expression of TNF-α, iNOS and IL-1β was analyzed by semi-quantitative RT-PCR (C) and quantitative real-time PCR (D). Data are means+SEM of five mice per group. ^{a}p<0.0001 vs saline group; ^{b}p<0.0001 vs the MPTP group,

FIG. 8 shows protection of dopaminergic neurons in MPTP-intoxicated mice by NaPB. Mice receiving NaPB (200 mg/kg body wt/day) from 3 h after the last injection of MPTP were sacrificed 7d after the last injection of MPTP followed by TH immunostaining of SNpc (A) and striatum (B), counting of TH-positive neurons in SNpc (C), quantification of TH-positive fibers in striatum (D), assay of neurotransmitters in striatum (E), and quantification of GSH in nigra (F). Data are means+SEM of eight mice per group. ^{a}p<0.0001 vs saline group; ^{b}p<0.0001 vs the MPTP group.

FIG. 9 shows protection of DA by FTI and GGTI and improvement of locomotor activities in MPTP-intoxicated mice. Mice receiving FTI, GGTI or the combination of the two via daily intraperitoneal injection from 3h after the last injection of MPTP were tested for motor functions (B, horizontal activity; C, total distance; D, number of movement; E, stereotypy) 7 d after the last injection of MPTP followed by measuring DA in striatum (A). Data are means SEM of eight mice per group. ^{a}p<0.001 vs saline; ^{b}p<0.001 vs MPTP; ^{c}p<0.05 vs saline; ^{a}p<0.05 vs MPTP.

FIG. 10 shows protection of striatal dopamine by NaPB and improvement of motor functions in a chronic MPTP mouse model of PD. A) Six to eight week old male C57BL/6 mice received 10 injections of MPTP (s.c.; 25 mg/kg body weight) together with 10 injections of probenecid (i.p.; 250 mg/kg body weight) for 5 weeks. Control group of mice received only saline. One group of mice were treated with NaPB (100 mg/kg body weight/d) via gavage from the 3rd injection of MPTP/probenecid and continued for 1 week thereafter. Mice were tested for different motor tasks [horizontal activity (C), number of movements (D), rearing (E), and stereotypy (F)] one week after the last injection of MPTP followed by measuring the level of DA in striatum (B). Data are means+SEM of eight mice per group. ^{a}p<0.001 vs saline; ^{b}p<0.001 vs MPTP; p<0.05 vs saline; ^{d}p<0.05 vs MPTP.

FIG. 11 shows the effect of NaPB on serum level of cholesterol in male C57/BL6 mice. Mice (6-8 wk old) were treated with NaPB (200 mg/kg body wt/d) and pravastatin (1 mg/kg body wt/d) separately via gavage for 7 d followed by quantification of cholesterol in serum using a simple fluorometric method. Results represent mean+SD of five mice per group (n=5). ^{a}_{p<}0.05 vs control.

FIG. 12 shows that NaPB attenuates MPP- induced expression of proinflatnmatory molecules in mouse microglial cells. Mouse BV-2 microglial cells were treated with different concentrations of NaPB for 6 h followed by stimulation with 1 μM. MPP under serum-free condition. After 5 h of stimulation, the mRNA expression of iNOS, IL-1β and TNF-α was monitored by semi-quantitative RT-PCR (A) and quantitative real-time PCR (C, iNOS; D, IL-1β; E, TNF-α). After 24 h of stimulation, the protein expression of iNOS was monitored by Western blot (B). Results was mean+SD of three different experiments. p<0.001 vs control; ^{b}p<0.00vs MPP.

FIG. 113 shows the time course of nigral activation of p21^{ras} and expression of iNOS and striatal loss of dopamine in MPTP-intoxicated mice. A) Mice were treated with NaPB (200 mg/kg body wt/d) via gavage from 1 d prior to MPTP injection. At different h of MPTP insult, nigral punches from mice were pooled together and analyzed for the activation of p21′ (A) and the expression of iNOS mRNA (B, RT-PCR; C, real-time PGR). Experiments were repeated three times each time using two animals in each group. ^{a}p<0.001 vs control (Oh)-iNOS. D) At different days of MPTP insult, dopamine was quantified in striata. Data are means±SEM of six mice per group. *p<0.05 vs control; “p<0.00.1 vs control.

FIG. 14 shows increased expression of CD11b and GFAP in ventral midbrain of MPTP-intoxicated mice is NaPB-sensitive. A) Mice were treated with NaPB (200 mg/kg body wt/d) from 3 h after the last injection of MPTP. Twenty-four h after the last injection of MPTP, ventral midbrain sections were immunostained for GFAP and CD11b. Results represent analyses of four nigral sections (two images per slide) from each of four mice. The mRNA expression GFAP and CD11b was analyzed by semi-quantitative RT-PCR (B) and quantitative real-time PCR (C). Data are means±SEM of five mice per group. ^{a}p<0.0001 vs saline group; ^{b}p<0.000/ vs the MPTP group.

FIG. 15 shows that NaPB improves motor functions in MPTP-intoxicated mice. Mice receiving NaPB (200 mg/kg body wt/day) from 3 h after the last injection of MPTP were tested for motor functions (A, rotorod; B, movement time; C, total distance; D, rest time; E, stereotypy counts; F, rearing; (3, horizontal activity) 7 d after the last injection of MPTP. Data are means±SEM of eight mice per group. ^{a}p<0.001 vs MPIP; ^{b}p0.05 vs MPTP.

### DETAILED DESCRIPTION

The inventors have made the surprising discovery that there is an association between small G-protein function and neurodegenerative disorders. More specifically, inhibition of p21^{ras} and/or p21^{rac} activation results in neurological anti-inflammatory and anti-oxidative effects. Inhibitors, such as sodium phenylbutyrate (NaPB), farnesyl transferase inhibitors (FTI), geranyigeranyl transferase inhibitors (GGTI), and combinations thereof, reduce activation of p21^{ras} and/or p21^{rac}.

### 1. Definitions

The terminology used herein is for the purpose of describing particular embodiments only and is not intended to be limiting. As used in the specification and the appended claims, the singular forms “a,” “and” and “the” include plural references unless the context clearly dictates otherwise.

For the recitation of numeric ranges herein, each intervening number there between with the same degree of precision is explicitly contemplated, For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

a. Therapeutically Effective Amount

“Therapeutically effective amount” as used herein may mean the amount of the subject agent or compound that will elicit the biological or medical response of a tissue, system, or subject that is being sought by the researcher, veterinarian, medical doctor or other clinician, and includes that amount of an agent or compound that, when administered, is sufficient to prevent development of or alleviate to some extent, one or more of the symptoms of the condition or disorder being treated.

### 2. Method of Treatment

Provided herein is a method of treating a neurodegenerative disorder in a subject in need thereof. The method comprises administering an inhibitor of small G-protein activation to the subject. The small G-protein may be p21^{rac}, p21^{ras}, or a combination thereof. The inhibitor may directly inhibit farnesol transferase and/or geranylgeranyl transferase. The inhibitor may be administered in a therapeutically effective amount.

a. Inhibitor of Small G-Protein Activation

The inhibitor of small G-protein may be any compound that directly or indirectly decreases or inhibits the activation of p21^{ras} and/or p21^{rac}. The inhibitor may directly or indirectly inhibit the activation of NF-κB. The inhibitor may directly or indirectly inhibit farnesyl transferase and/or geranylgeranyl transferase. The inhibitor may exert anti-inflammatory and/or antioxidative effects via one or more of its inhibitory function(s). The inhibitor may protect substantia nigra (“nigra” or “nigral”) reduced glutathione, attenuate nigral activation of NF-kB, inhibit nigral expression of proinflammatory molecules, and/or suppress nigral activation of glial cells. For example, the inhibitor may be a sodium phenylbutyrate (NaPB), an inhibitor of farnesyl transferase (FTI), an inhibitor of gerany eranyl transferase (GGTI), or a combination thereof.

(1) Sodium Phenylbutyrate (NaPB)

NaPB is an FDA-approved drug that has been used against urea cycle disorders in humans. The NaPB may suppress the activation of NF-kB, inhibit the expression of proinflammatory molecules, and/or attenuate the production of reactive oxygen species (ROS) from activated microglia. NaPB may modulate the mevalonate pathway and/or inhibit the activation of p21^{ras} and p21^{rac}. The NaPB may inhibit activation of p21^{ras} and p21^{rac}. Nigral activation of p21^{ras} and p21^{rac} may be inhibited by NaPB. Nigral expression of (proinflammatory molecules may be suppressed or inhibited by NaPB. NaPB may increase the level of the antioxidant, glutathione (GSH) and/or protect the nigrostriatal axis.

(2) :Farnesyl Transferase (FTI)

FTI may be any inhibitor of farnesyl transferase. The FTI may be a naturally occurring compound or a synthetically produced compound. An example of a naturally occurring FTI is manumycin, which is isolated form Streptomyces sp. A drug library may be screened for FTIs. Examples of FTI identified by this approach include SCH6636, a tricyclic inhibitor, and R115777, a nonpeptidomimetic inhibitor. The FTI may be Tipifarnib (6-[amino(4-chlorophenyl)(1-methyl-1H-imidazol-5-yl)methyl]-4-(3 -chlorophenyl)-1-methylquinolin-2(1H)-one) or Lonafarnib (4-(2-(4-(8-Chloro-3,10-dibromo-6,11-dihydro-5H-benzo(5,6)cyclohepta(1,2-b)pyridin-11-yl)-1-piperidinyl)-2-oxoethyl)-1-piperidinecarboxamide). Other examples of FTI inhibitors include those described in Brunner et al., Cancer Res., 63:5656-5668 (2003) and Vogt et al., Oncogene 13: 1991-1999 (1996), each of which is hereby incorporated by reference in its entirety.

(3) Geranylgeranyl Transferase (GGTI)

GGTI may be any inhibitor of geranylgeranyl transferase. The GGTI may be a naturally occurring compound or a synthetically produced compound. The GGTI may inhibit protein modification and/or block membrane association of Ral, Rho, and Rap subfamilies. Examples of GGTI compounds include GGTI-298 (Miguel et al., Cancer Res., 57:1846-1850 (1997)), GGTI-2154 (Sun et al., Cancer Res., 63:8922-8929 (2003)), GGTI-2 166 (Woo et al., Biochem. Pharmacol., 69:87-95 (2005)), GGTI-286 (Bredel et al, Neurosurgery, 43:124-131 (1998)), GGTI-2166 (Sun et al., Cancer Res., 59:4919-4926 (1999)), and GGTI-DL¹45 (Peterson et al., Biol. Chem., 281:12445-12450 (2006)).

b. Formulations and Administration

The inhibitor may be formulated for parenteral administration by injection, e.g., by bolus injection or continuous infusion. Formulations for injection may be presented in unit dosage form, e.g., in ampules or in multi-dose containers, with an added preservative. The inhibitor may take such a form as a suspension, solution, or emulsion in oily or aqueous vehicles, and may contain formulating agents such as suspending, stabilizing and/or dispersing agents. The inhibitor may be prepared for oral administration. The inhibitor may be suitably formulated to give controlled release of the inhibitor. For buccal administration, the inhibitor may take the form of tablets or lozenges formulated in conventional manner. For administration by inhalation, the inhibitor may be delivered in the form of an aerosol spray presentation from pressurized packs or a nebuliser, with the use of a suitable propellant. In the case of a pressurized aerosol, the dosage unit may be determined by providing a valve to deliver a metered amount,

In general, the inhibitor will be administered in a therapeutically effective amount by any of the accepted modes of administration for agents and compounds that serve similar utilities. The actual amount of the PKG-effector agent and/or the compound for combination treatment of this invention will depend upon numerous factors such as the severity of the neurodegenerative disorder to be treated, the age and relative health of the subject, the potency of the compound used, the route and form of administration, and other factors. The inhibitor can be administered more than once a day, preferably once or twice a day. Therapeutically effective amounts of the inhibitor may range from approximately 0.05 mg to 10 g per kilogram body weight of the subject per day. For example, therapeutically effective amounts of the inhibitor may range from 0.1 mg to 10 mg per kilogram body weight of the subject per day, from 0.1 mg to 10 mg per kilogram body weight of the subject per day, from 0.5 mg to 10 mg per kilogram body weight of the subject per day, from 1 mg to 10 mg per kilogram body weight of the subject per day, from 3 mg to 10 mg per kilogram body weight of the subject per day, from 5 mg to 10 mg per kilogram body weight of the subject per day, from 7 mg to 10 mg per kilogram body weight of the subject per day, from 9 mg to 10 mg per kilogram body weight of the subject per day, from 1 mg to 5 mg per kilogram body weight of the subject per day, or from 2 mg to 7 mg per kilogram body weight of the subject per day.

c. Small G.-Protein

The small G-protein may be any G-protein associated with the production of ROS and/or inflammation. The small G-protein may directly or indirectly control the production of proinflammatory molecules and/or ROS glial cells, which may be activated glial cells. The small G-protein may be activated in the substantia nigra of subjects having a neurodegenerative disorder. The small G-protein may be p21^{ras} or p21^{rac}, for example.

d. Neurodegenerative Disorder

The neurological disorder may be any central nervous system (CNS) or peripheral nervous system (PNS) disease that is associated with neuronal or glial cell defects including but not limited to neuronal loss, neuronal degeneration, neuronal demyelination, gliosis (i.e., astrogliosis), or neuronal or extraneuronal accumulation of aberrant proteins or toxins (e.g., β-amyloid, or α-synuciein). The neurological disorder can be chronic or acute. Examples of various chronic and acute neurological diseases include Parkinson's disease, Alzheimer's disease, myasthenia gravis, multiple sclerosis, microbial infections, head trauma and stroke, Pick's disease, dementia with Lewy bodies, Huntington disease, chromosome 13 dementias, Down's syndrome, cerebrovascular disease, Rasmussen's encephalitis, viral meningitis, NPSLE, amyotrophic lateral sclerosis, Creutzfeldt-Jacob disease, Gerstrnann-Straussler-Scheinker disease, transmissible spongiform encephalopathies, ischemic reperfusion damage (e.g. stroke), brain trauma, microbial infection, chronic fatigue syndrome, Mild Cognitive Impairment; and movement disorders (including ataxia, cerebral palsy, choreoathetosis, dystonia, Tourette's syndrome, kernicterus); tremor disorders, leukodystrophies (including adrenoleukodystrophy, metachromatic leukodystrophy, Canavan diseaseAlexander disease, Pelizaeus-Merzbacher disease); neuronal ceroid lipofucsinoses; ataxia telangectasia; and Rett Syndrome.

e. Subject

The subject may be a mammal. The mammal may be a human. The human may be at risk of developing a neurological disorder. The subject may be a human already diagnosed with a neurological disorder.

The present invention has multiple aspects, illustrated by the following non-limiting examples.

### EXAMPLES

### Example 1

NaPB Attenuates the Expression of iNOS and Proinflammatory Cytokines in Activated Mouse Microglia and Human Astroglia

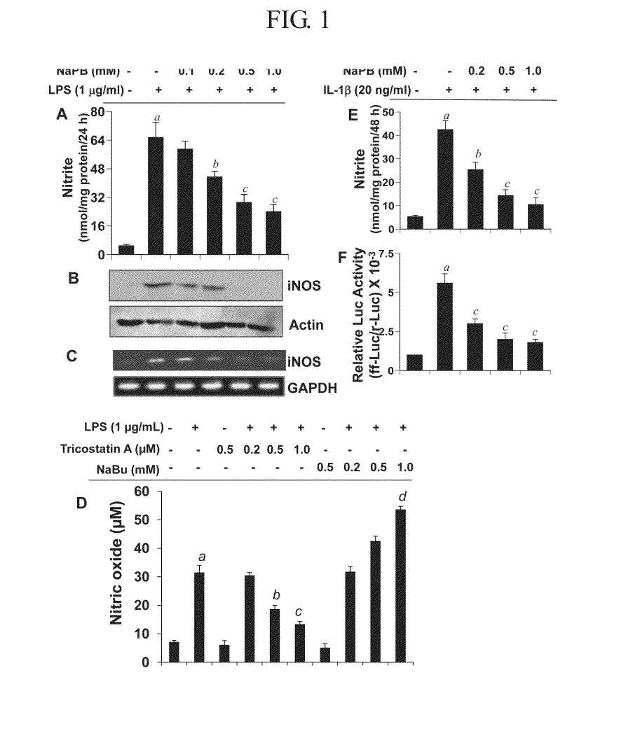

Activated microglia and astroglia are known to produce excessive amount of NO having the potential of damaging neurons in neurodegenerative disorders. We investigated the effect of NaPB on the expression of iNOS in microglia. LPS is a prototype inducer of various proinflammatory molecules in different cell types including mouse microglia. Therefore, primary mouse microglia preincubated with different doses of NaPB for 6 h were stimulated with LPS under serum-free condition. Although at lower concentration (100 μM), NaPB was not effective in inhibiting the production of NO, at higher concentrations (>200 μM), NaPB markedly suppressed LPS-induced production of NO in microglia (FIG. 1A). However, 1 or 2 h preincubation of microglia with NaPB was not sufficient to exhibit NO inhibition (data not shown). Because NaPB is a known inhibitor of histone deacetylase (HDAC), we investigated if other inhibitors of HDAC also shared this property. We used trichostatin A (TSA) and sodium butyrate (NaBu) for this purpose. Although similar to NaPB, TSA attenuated LPS-induced production of NO, ⁻NaBu stimulated the production of NO in LPS-stimulated microglial cells (FIG. 1D) suggesting that all HDAC inhibitors do not have anti-inflammatory property. To understand the mechanism for NaPB-mediated suppression of NO production, we investigated the effect of NaPB on protein and mRNA expression of iNOS in microglia. As evident from Western blot (FIG. 1B) and RT-PCR (FIG. 1C), NaPB dose-dependently inhibited LPS-induced protein and mRNA expression of iNOS in microglia. (3-(4,5-dimethylthiazol-2-yl)-2,5-diphenyltetrazolium bromide (MTT) results show that NaPB was not toxic to microglia at any of the concentrations tested (data not shown) suggesting that the inhibitory effect of NaPB on microglial expression of iNOS was not due to any change in cell viability. In addition to producing NO, activated microglia also secrete a broad range of proinflammatory molecules. Therefore, we examined if NaPB was capable of suppressing the expression of proinflatrunatory cytokines in primary mouse microglia. Similar to the inhibition of iNOS, NaPB dose-dependently inhibited the production of TNF-α and IL-1β protein in activated microglia (Table-1).

TABLE 1
Attenuation of Proinflammatory Cytokine Production by NaPB in Primary Mouse Microglia

| | Treatment | | | | | |
|---|---|---|---|---|---|---|
| Cytokine (ng/mg | | | LPS + NaPB | LPS + NaPB | LPS + NaPB | NaPB |
| protein/24 hr) | Control | LPS | (0.1 mM) | (0.2 mM) | (0.5 mM) | (0.5 mM) only |
| TNF-α | 0 | 18.4 ± 3.1 | 16.8 ± 1.5 | 12.7 ± 0.8 | 8.6 ± 1.1^{a} | 0 |
| IL-1β | 0 | 11.2 ± 2.9 | 9.9 ± 1.4 | 7.5 ± 1.1 | 5.3 ± 0.6^{a} | 0 |

Primary microglia preincubated with differnt concentrationis of NaPB for 6 hrs were stimulated with LPS for 24 hrs followed by quantification of TNF-α and IL-1β in supernatants by ELISA. Results are mean ± SD of three independent experiments.
^{a}p < 0.001 vs LPS.

Next, we examined if NaPB could suppress the expression of iNOS in human brain cells. Astroglia are the major glial cells in the CNS and astroglial activation also plays a role in various neurodegenerative disorders. We have found that IL-1β is the only cytokine that induces iNOS in primary human astroglia. Consistently, IL-1β induced the production of nitrite (FIG. 1E) and the activation of human iNOS promoter (FIG. 1F) in primary astroglia isolated from human fetal brains. interestingly, NaPB markedly inhibited IL-1β-induced production of NO (FIG. 1E) and the activation of iNOS promoter (FIG. 1F) in human astroglia.

### Example 2

### NaPB Inhibits Microglial Activation of NF-κB

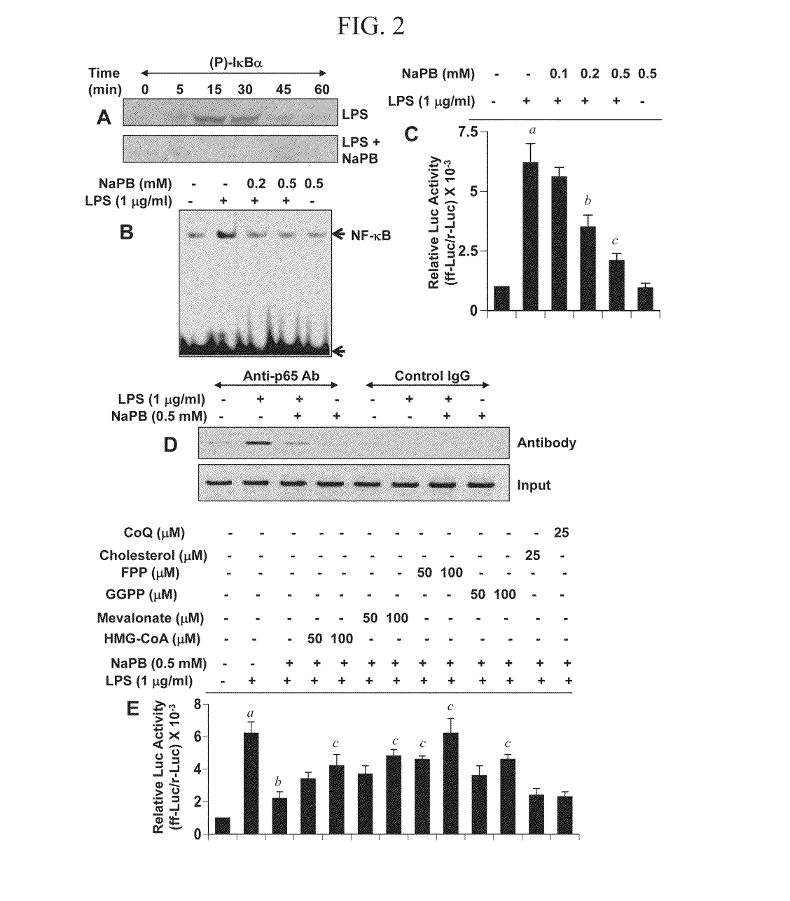

LPS and other inflammatory stimuli including MPP+ induce iNOS expression via activation of NF-κB. Because NaPB attenuated the expression of iNOS in glial cells, we examined the effect of NaPB on the activation of NF-κB. Activation of NF-κB was monitored by IκBαphosphorylation, DNA binding activity by EMSA and transcriptional activity by reporter assays. As expected, treatment of BV-2 microglial cells with LPS resulted in time-dependent increase in phospho-IκBα (FIG. 2A). However, NaPB inhibited LPS-induced phosphorylation of IκBαsuggesting that NaPB functions upstream of IκBα phosphorylation. Accordingly, LPS induced the DNA-binding activity of NF-κB in microglial cells, which was inhibited by NaPB (FIG. 2B). We then tested the effect of NaPB on the transcriptional activity of NF-κB. As expected, LPS induced NF-κB-dependent transcription of luciferase (FIG. 2C). Consistent to the effect of NaPB on the phosphorylation of IκBα and the DNA binding activity of NF-κB, NaPB also suppressed the transcriptional activity of NF-κB in a dose-dependent manner in LPS-stimulated microglia (FIG. 2C).

Mouse iNOS promoter harbors two NF-κB binding sites—distal (nucleotides-971 to -962) and proximal (nucleotides-85 to -76) At first, we employed chromatin immunoprecipitation (ChIP) assay to study the recruitment of RelA p65 to each of these two NF-κB binding sites. After immunoprecipitation of LPS-stimulated microglial chromatin fragments by antibodies against p65, we were able to amplify 307 bp fragments flanking the proximal NF-κB element (FIG. 2D). However, after several attempts, we failed to detect any amplification product spanning the distal NF-κB binding site (data not shown). These results suggest that LPS induced the recruitment of p65 to the proximal NF-κB-binding site of the mouse iNOS promoter in microglia. Therefore, next we examined the effect of NaPB on the recruitment of p65 to the proximal NF-κB binding site of the iNOS promoter. Consistent to the inhibition of iNOS mRNA expression, NaPB inhibited the recruitment of p65 to the iNOS promoter in LPS-stimulated microglia (FIG. 2D). On the other hand, no amplification product was observed in any of the immunoprecipitates obtained with control IgG (left four lanes of FIG. 2D) suggesting the specificity of these interactions. These results also suggest that NaPB interferes with the recruitment of NF-κB to the iNOS promoter.

### Example 3

### Intermediates of the Mevalonate Pathway Reverse the Inhibitory Effect of NaPB on Microglial NF-κB Activation

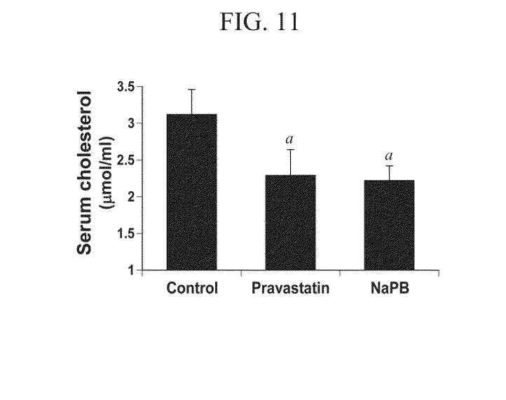

The requirement of at least 6 h of preincubation of cells with NaPB to see its anti-inflammatory effect suggests that metabolite(s) sensitive to NaPB may be involved in the process. Intermediates of the mevalonate pathway play a role in the expression of iNOS and proinflammatory cytokines in glial cells. End product of the mevalonate pathway is cholesterol and therefore, we investigated if NaPB had any effect on the level of cholesterol in vivo in mice. Interestingly, after 7 d of oral feeding at a dose of 200 mg/kg body wt/d, NaPB reduced the level of cholesterol in serum of mice by about 30%; and this reduction was comparable to that (˜29%) by the so-called cholesterol-lowering drug pravastatin (FIG. 11). These results are important as it suggests that NaPB may be used to lower cholesterol in patients with hypercholesterolemia.

Next, we examined the role of different members of the mevalonate pathway in NF-κB inhibitory effect of NaPB. Interestingly, HMG-CoA, mevalonate, geranylgeranyl pyrophosphate (GGPP), and farnesyl pyrophosphate (FPP) abrogated the inhibitory effect of NaPB on the activation of NF-κB (FIG. 2E) in microglial cells. On the other hand, cholesterol and coenzyme Q (end products of the mevalonate pathway) had no effect on NaPB-mediated inhibition of NF-κB activation (FIG. 2E). These results suggest that depletion of intermediary products rather than end products of the mevalonate pathway is responsible for the observed anti-inflammatory effect of NaPB.

### Example 4

### NaPB Inhibits MPP⁺-induced Expression of Microglial Proinflammatory Molecules

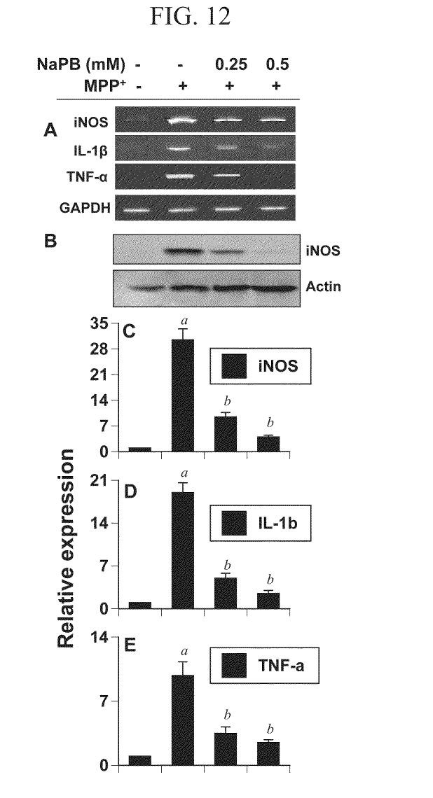

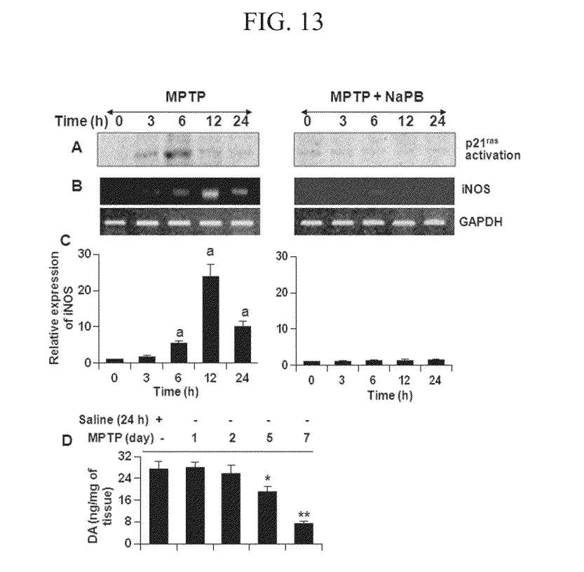

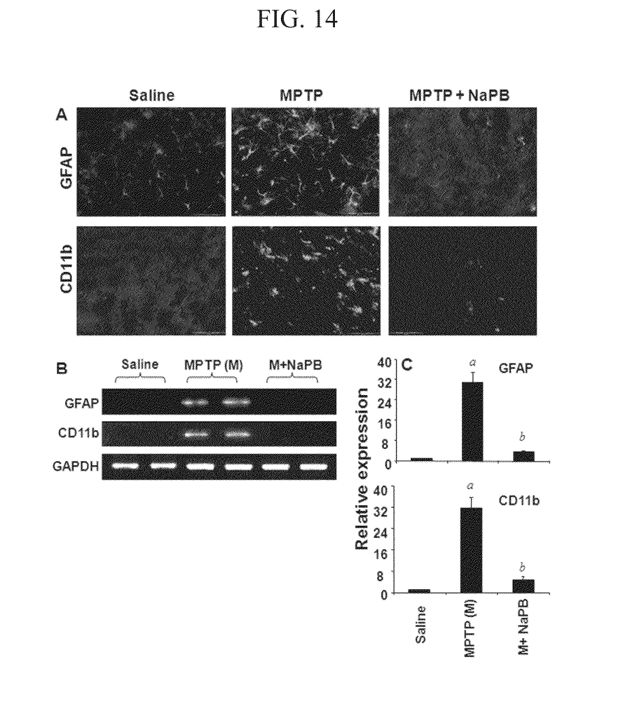

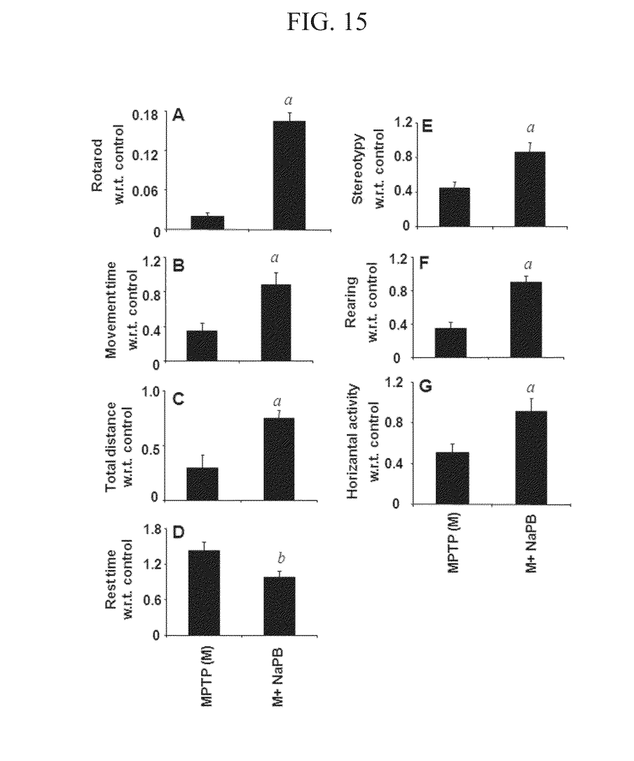

Glial inflammation is a critical component of PD pathogenesis which is mirrored in MPTP mouse models. The neurotoxic effect of MPTP depends on its conversion into MPP+. In glial cells, MAO-B converts MPTP to MPP+, which then leads to glial activation. MPP+ is able to stimulate the microglial expression of inflammatory molecules including NO, TNF-α, IL-1β, and IL-6. Although, a well characterized receptor for MPP+ was not known in microglial cells until recent past, low dose of MPP+ stimulates microglial activation via the engagement of cysteinyl leukotriene receptor (CysLT1R), MPP+ dependent activation of CysLT1R and its subsequent translocation from plasma membrane to cytosol plays a critical role in the process of microglial activation. Therefore, we investigated MPP+ could induce the expression of proinflammatory molecules microglia and if NaPB suppressed such induction. RT-PCR (FIG. 12A), western blot (FIG. 12B) and real-time PCR (FIG. 12C) analysis showed that MPP+ alone induced the expression of iNOS mRNA and protein and that NaPB suppressed MPP+-induced iNOS mRNA and protein expression (FIG. 12A-C) microglial cells. MPP+ also markedly induced the mRNA expression of IL-1β and TNF-α microglial cells (FIGS. 12A, 12D & 12E). However, NaPB inhibited MPP+-induced expression of TNF-α and IL-1β mRNAs in a dose-dependent manner (FIG. 12A, 12D 12E) suggesting that NaPB is capable of inhibiting MPP+-induced expression of microglial proinflammatory molecules.

### Example 5

### NaPB Inhibits the Production of Reactive Oxygen Species (ROS) from Activated Microglia

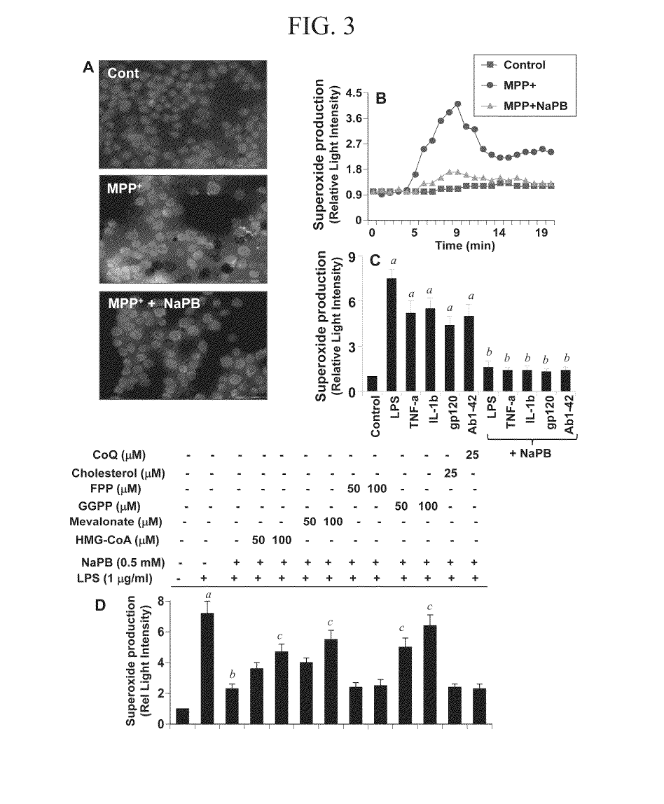

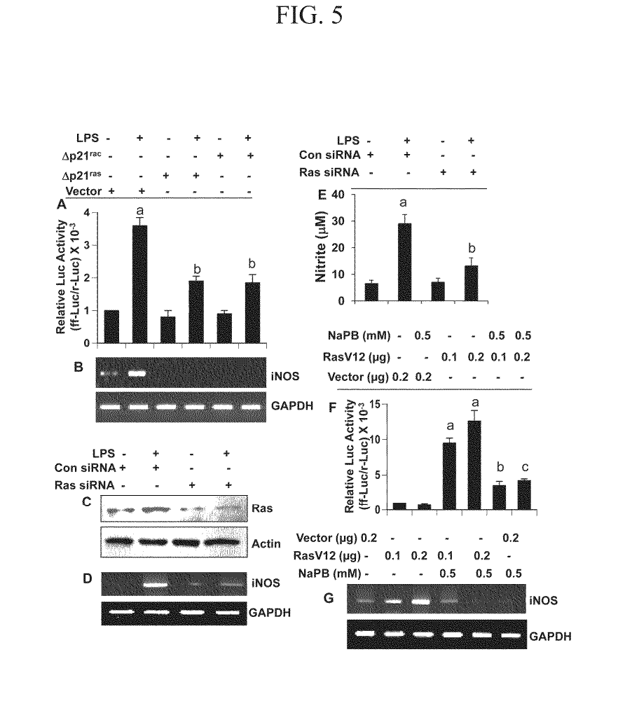

Oxidative stress plays an important role in the pathogenesis of various neurodegenerative diseases including PD. We examined if Parkinsonian toxin MPP+ induced the production of ROS from microglia and if NaPB could attenuate such ROS production. To monitor the generation of intracellular ROS in BV-2 microglial cells, we used a cell-permeant fluorescent probe. As seen in FIG. 5A, MPP+ markedly induced the generation of ROS within 15 min of stimulation. However, NaPB strongly inhibited MPP-i--induced production of intracellular ROS (FIG. 3A). NADPH oxidase is an important ROS (superoxide radicals)-producing molecule in response to different inflammatory stimuli. Accordingly, MPP+ also induced the production of superoxide from microglial cells in a time-dependent manner (FIG. 3B). The production of superoxide was observed as early as 5 min of stimulation, which peaked at 10 min (FIG. 3B). Consistent to the inhibition of endogenous ROS (FIG. 3A), NaPB attenuated MPP+-induced production of superoxide (FIG. 3B). Because various stimuli and neurotoxins are capable of producing ROS, we examined if NaPB could suppress the production of ROS in response to different inflammatory stimuli. As expected, LPS, TNF-α, IL-1β, HIV-1 gp120, and fibrillar Aβ1-42 peptides induced the production of superoxide in microglial cells (FIG. 3C). However, NaPB knocked down LPS-, TNF-α-, IL-1β, gp120-, and Aβ-induced production of superoxide in microglia (FIG. 3C). These results suggest that NaPB could be used as an antioxidant.

Because NaPB exhibited anti-inflammatory activity via modulation of mevalonate metabolites (FIG. 2E), we investigated if mevalonate metabolites could reverse the antioxidant effect of NaPB, Interestingly, HMG-CoA, mevalonate and GGPP, but not FRP, abolished the inhibitory effect of NaPB on the production of superoxide in microglial cells (FIG. 3D). On the other hand, cholesterol and coenzyme Q had no effect on NaPB-mediated inhibition of superoxide production (FIG. 3D). These results suggest that depletion of intermediary products rather than end products of the mevalonate pathway is also responsible for the antioxidant effect of NaPB. Furthermore, due to that geranylgeranylation is required fir the activation of p21^{rac} and that farnesylation is needed for the activation of p21^{ras}, these results suggest that p21^{rac}, but not p21^{ras}, may be involved in ROS generation.

### Example 6

### NaPB Mediates its Anti-Inflammatory Effects through Inhibition of p21^{ras} and p21^{ras} Activation

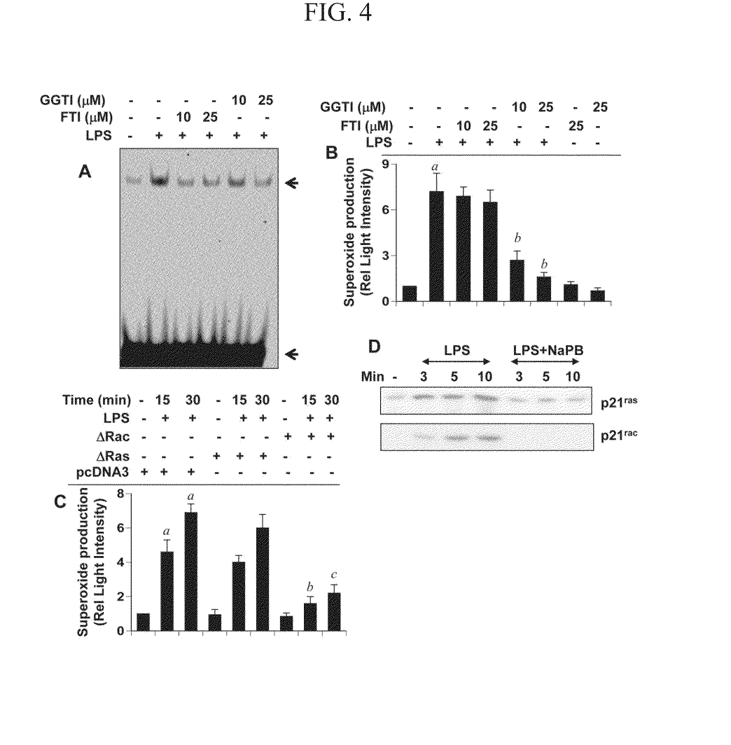

Reversal of anti-inflammatory and antioxidant effects of NaPB by intermediates, but not end products, of the mevalonate pathway, suggest a possible involvement of farnesylation and/or geranyigeranylation reactions in inflammation and oxidative stress. Because farnesylation and geranylgeranylation are required for activation of p21^{ras} and p21^{rac}, respectively, we examined the effect of farnesyltransferase inhibitor (FTI) and geranylgeranyltransferase inhibitor (GGTI) on NF-κB activation and superoxide production in microglial cells. Inhibition of LPS-induced activation of NF-κB by both FTI and GGTI (FIG. 4A) suggests that both p21^{ras} and p21^{rac} could be involved in microglial activation of NF-κB. To confirm the involvement of p21^{ras} and p21^{rac}, we examined the effect of dominant-negative mutants of p21^{ras} (Δp21^{ras}) and p21^{rac} (Δp21^{rac}) on LPS-induced activation of NF-κB and expression of iNOS. LPS induced the transcriptional activity of NF-κB (FIG. 5A) and the expression of iNOS mRNA (FIG. 5B) in empty vector-transfected microglial cells. However, both Δp21^{ras} and Δp21^{rac} suppressed LPS-induced activation of NF-κB (FIG. 5A) and expression of iNOS (FIG. 5B). This was further corroborated by siRNA knockdown of p21^{ras}. Ras siRNA decreased the protein expression of p21^{ras} (FIG. 5C) and inhibited LPS-induced expression of iNOS tuRNA (FIG. 5D) and production of nitrite (FIG. 5E). Accordingly, RasV12, a constitutively-active mutant of p21^{ras}, alone induced the activation of NE-κB (FIG. 5F) and the expression of iNOS mRNA (FIG. 5G) suggesting that activation of p21^{ras} alone is sufficient for the activation of NF-κB and the expression of iNOS. NaPB, in this instance as well, attenuated RasV12-induced activation of NF-κB and expression of iNOS (FIG. 5F-G).

Although both FTI and GGTI inhibited the activation of NF-κB, only (iGTI, but not inhibited the production of superoxide from LPS-stimulated microglial cells (FIG. 4B) suggesting that p21^{rac}, but not p21^{ras}, could be involved in the generation of superoxide. To further confirm this finding, we examined the effect of dominant-negative mutants of p21^{ras} (Δp21^{ras}) and p21^{rac} (Δp21^{rac}) on LPS-induced production of superoxide. Consistently, Δp21^{rac}, but not Δp21^{ras}, knocked down ITS-induced production of superoxide in microglial cells. This was not seen in empty vector (pcDNA3) controls (FIG. 4C). Next, we examined the effect of NaPB on the activation of p21^{ras} and p21^{rac}. Marked activation of p21^{ras} and p21^{rac} was observed within minutes of ITS stimulation (FIG. 4D). However, NaPB markedly suppressed LPS-induced activation of both p21^{ras} and p21^{rac} in microglial cells (FIG. 4D). These results suggest that NaPB attenuates the expression of proinfiammatory molecules and the production of ROS in microglia probably by suppressing the activation of p21^{ras} and p21^{rac}.

### Example 7

### Oral Administration of NaPB Attenuates the Activation of p21^{ras and p}21^{rac} In Vivo in the Nigra of MPTP-Intoxicated Mice

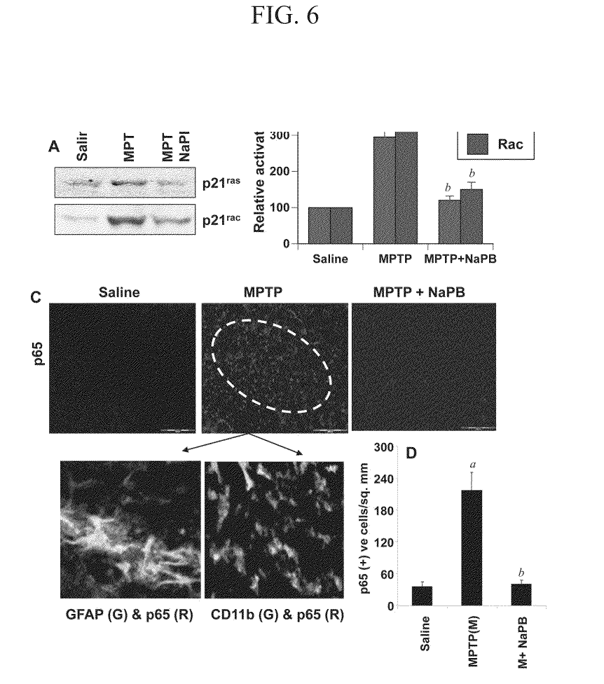

Because NaPB inhibits the activation of p21^{ras} and p21^{rac} in microglia, we examined if NaPB was capable of suppressing the activation of these small G proteins in vivo in the nigra of MPTP-insulted mice, an animal model of PD. It is clearly evident from FIGS. 6A & 6B that MPTP intoxication markedly induced the activation of p21^{ras} and p21^{rac} in the nigra compared to saline treatment. However, mice that were treated with NaPB (200 mg/kg body wt/day through gavage from l d prior to the MPTP intoxication exhibited much decreased activation of both p21^{ras} and p21^{rac} (FIGS. 6A & 6B) suggesting that oral NaPB is capable of inhibiting the activation of p21^{ras} and p21^{rac} in vivo in the nigra.

### Example 8

### NaPB Inhibits the Activation of NF-κB In Vivo in the Nigra of MPTP-Intoxicated Mice

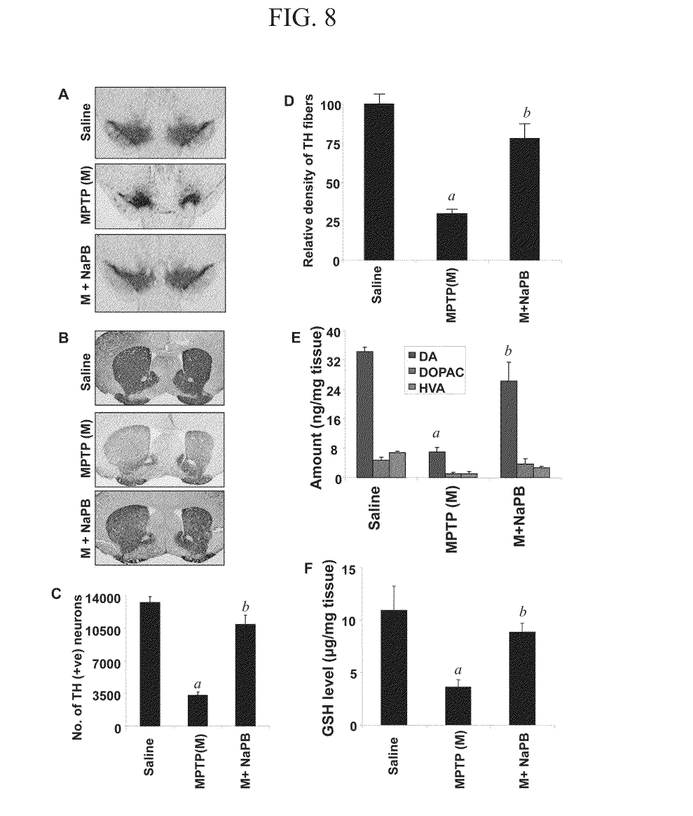

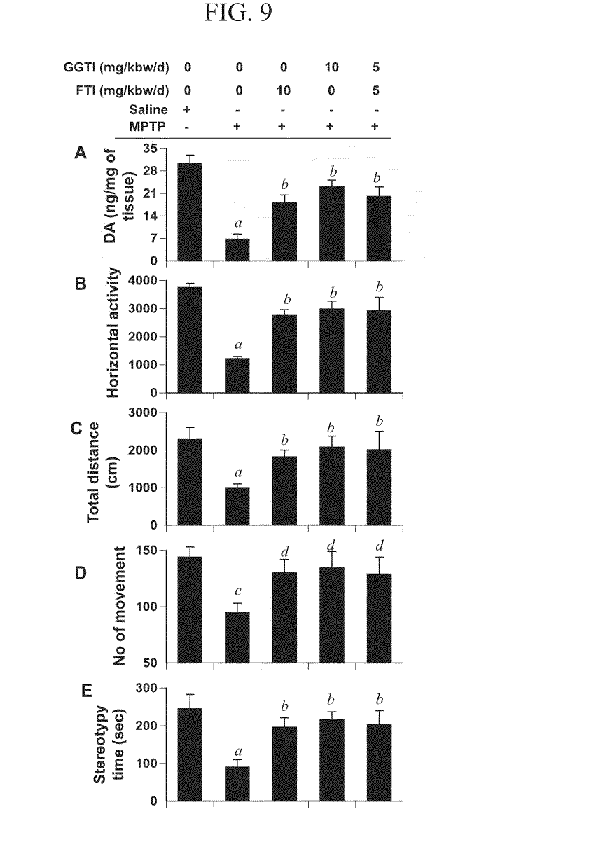

NaPB inhibited the activation of NF-κB in glial cells (FIG. 2), we examined if NaPB was capable of suppressing the activation of NF-κB in vivo in the nigra of MPTP-intoxicated mice. It is clearly evident from FIGS. 6C and 6D that MPTP intoxication markedly induced the expression of RelA p65 in the SNpc as compared to saline treatment. Double-label immunofluorescence analysis indicates that p65 was principally expressed in CD11b-positive microglia and CiFAP-positive astroglia (FIG. 6C). Next, mice were treated with NaPB (200 mg/kg body wt/day) via gavage from 3 h after the last injection of MPTP and the activation of NF-κB was examined 24 h after the last injection of MPTP. As evident from FIGS. 6C and 8D, NaPB markedly inhibited the level of p65 in vivo in the SNpc of MPTP-intoxicated mice.

### Example 9

### NaPB Inhibits the Expression of Proinflammatory Molecules In Vivo in the Nigra of MPTP-Intoxicated Mice

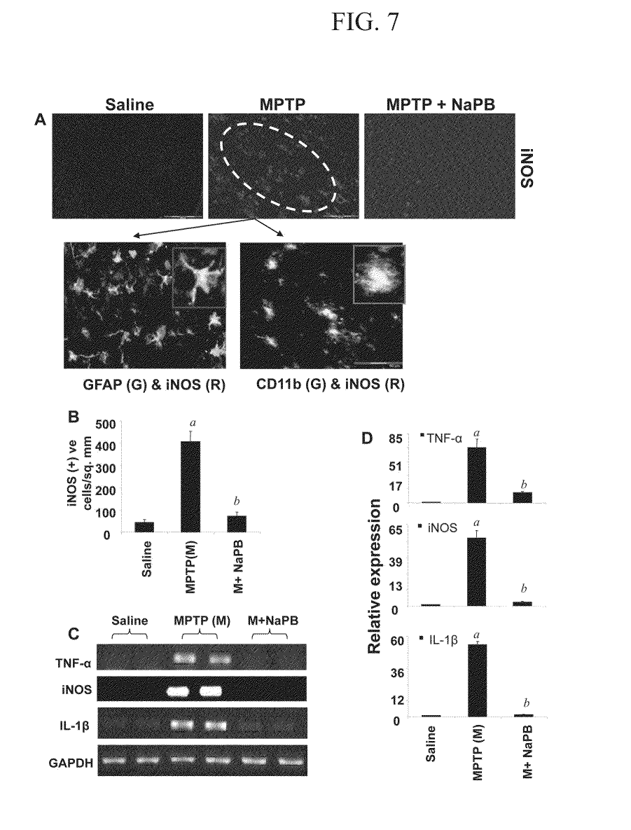

Inflammation plays a role in the loss of dopaminergic neurons in PD and its animal model, Because NaPB inhibited the expression of proinflammatory molecules in glial cells and suppressed the activation of small G proteins (p21^{ras} and p21^{rac}) and NF-κB in vivo in the nigra of MPTP-intoxicated mice, we examined if NaPB was able to suppress the expression of iNOS in vivo in the SNpc of MPTP-insulted mice. Immunofluorescence analysis for iNOS in ventral midbrain sections shows that MPTP intoxication led to marked increase in nigral iNOS protein expression and that iNOS co-localized with GFAP-positive astroglia and CD11b-positive microglia (FIG. 7A). However, oral administration of NaPB suppressed MPTP-induced expression of iNOS protein (FIG. 7A-B). As shown by semi-quantitative RT-PCR (FIG. 7C) and quantitative real-time PCR (FIG. 7D) experiments, MPTP intoxication led to marked increase in mRNA expression of iNOS, and TNF-α in the nigra. However. NaPB strongly inhibited MPTP-induced expression of these proinflammatory molecules in vivo in the nigra (FIG. 7C-D). These results suggest NaPB can inhibit the expression of proinflammatory molecules in vivo in the SNpc of MPTP-intoxicated mice.

### Example 10

### Glial Inflammation/Activation Appears Before neuronal Loss in MPTP Mouse Model of PD

While microglial activation occurs much earlier to neuronal death in MPTP mouse model, activation of glia, specifically microglia, is secondary to neurodegeneration. Therefore, in order to delineate whether glial activation-associated events happen before or after the loss of dopaminergic neurons, we performed a time-course study to monitor nigral activation of p21^{ras} and expression of iNOS and striatal loss of dopamine in MPTP-intoxicated mice, Micropunches from the nigra were used for monitoring p21^{ras} and iNOS. The activation of p21^{ras} began as early as 3 h after the last injection of MPTP, peaked at 6 h and decreased afterwards until the duration (24 h) of the study (FIG. 13A). On the other hand, the expression of iNOS mRNA as revealed by RT-PCR (FIG. 13B) and real-time PCR (FIG. 13C) was visible as early as 6 h and maximum at 12 h. As expected from results above, NaPB treatment markedly inhibited the activation of p21^{ras} and the expression of iNOS mRNA in vivo the nigra at all time points tested (FIG. 13A-C). In contrast to the activation of p21^{ras} and the expression of iNOS, we did not notice any loss of striatal dopamine within 24 h of MPTP intoxication (FIG. 13D). Significant loss of dopamine was observed on day 5 and it was maximum on day 7 (FIG. 13D). These results suggest that glial inflammation/activation appears before neuronal loss in MPTP mouse model of PD.

### Example 11

### NaPB Inhibits the Activation of Glial Cells In Vivo in the Nigra of MPTP-Intoxicated Mice

Recently activation of glial cells is being considered as a pathological hallmark in PD and other neurodegenerative disorders. Increased expression of CD11b, the beta-integrin marker of microglia, represents microglial activation during neurodegenerative inflammation. Similarly, upon activation, astrocytes also express enhanced level of GFAP, which is considered as a marker protein for astrogliosis. We investigated if NaPB could attenuate MPTP-induced activation of glial cells in vivo in the nigra of mice. As evident from immunofluorescence analyses of CD11b and GFAP in ventral midbrain sections (FIG. 14A), MPTP intoxication led to marked increase in nigral CD11b and GFAP protein expression, However, oral treatment of MPTP-intoxicated mice with NaPB led to the inhibition of GFAP and CD11b protein expression (FIG. 14A). It is clearly evident from semi-quantitative RT-PCR in FIG. 14B and real-time PCR in FIG. 14C that MPTP intoxication led to marked increase in mRNA expression of both CD11b and GFAP in the nigra. However, similar to the inhibition of proinflammatory molecules, NaPB suppressed MPTP-induced expression of CD11b and GFAP in vivo in the nigra (FIG. 14B-C). These results suggest that NaPB is capable of attenuating glial activation in vivo in the nigra of MPTP-intoxicated mice.

### Example 12

### NaPB Protects Against MPTP-Induced Neurodegeneration

MPTP-intoxication led to approximately 75% loss of SNpc TH-positive neurons (FIGS. 8A & C) and 70% reduction of striatal TEL ODs (FIGS. 8B & D) compared with saline-injected controls. However, in MPTP-injected mice treated with NaPB, less reduction in SNpc TH-positive neurons and striatal TH ODs was observed (FIG. 8A-D), Next to determine whether NaPB protects against biochemical deficits caused by MPTP, we quantified levels of dopamine (DA) and two of its metabolites, dihydroxyphenytacetic acid (DOPAC) and hornovanillic acid (HVA), in the striata 7 days after the MPTP treatment. As evident from FIG. 8E, MPTP intoxication led to about 76% decrease in striatal DA compared to striata of saline-injected mice. in contrast, MPTP-i.ntoxicated animals that received NaPB showed only 20-25% decrease in striatal dopamine (FIG. 8E).

Because NaPB inhibited the activation of p21^{rac} and the production of ROS from microglial cells and attenuated the activation of p21^{rac}in vivo in the nigra of MPTP-insulted mice, we examined the effect of NaPB on nigral redox state in MPTP-insulted mice. Reduced glutathione (GSH) is the master anti-oxidant, which protects all cells including dopaminergic neurons from oxidative attack. We monitored the level of nigral GSH by HPLC. As expected, MPTP intoxication led to approximately 72% loss of GSH (FIG. 8F), However, after NaPB treatment, MPTP-intoxicated mice showed only 18% loss of nigral GSH (FIG. 8F) suggesting that NaPB is capable of improving the nigral redox state in MPTP-intoxicated mice.

### Example 13

### NaPB Improves Locomotor Functions in MPTP-Intoxicated Mice

The ultimate therapeutic goal of neuroprotection is to decrease functional impairment. Therefore, to examine whether NaPB protects not only against structural and neurotransmitter damage but also against functional deficits caused by MPTP, we monitored locomotor and open-field activities. MPTP injection caused marked decrease in rotorod performance (FIG. 15A), movement time (FIG. 15B), total distance (FIG. 15C), stereotypy (FIG. 15E), rearing (FIG. 15F), and horizontal activity (FIG. 15G). On the other hand, MPTP increased the rest time (FIG. 15D). Interestingly, NaPB significantly improved MPTP-induced hypolocomotion (FIG. 15A-G).

### Example 14

### Is Inhibition of p21^{ras} farnesylation and/or p21^{rac} geranylgeranylation sufficient to protect the Nigrostraitum against MPTP Toxicity?

In addition to inhibiting farnesylation of p21^{ras} and geranylgeranylation of p21^{rac}, NaPB exhibits many other biological functions including histone deacetylase (HDAC) inhibition, chemical chaperoning upon endoplasmic reticulum (ER) stress and ammonia scavenging in urea cycle disorders, which could be responsible for the protection of nigrostriatum from MPTP neurotoxicity. Therefore, we investigated if inhibition of either p21^{ras} or p21^{rac} alone was sufficient for the protection of the nigrostriatum. MPTP-intoxicated mice received FTI and GGTI, either separately at 10 mg/kg body wt/d or together at 5 mg/kg body wt/d, via i.p. injection from 3 h after the last injection of MPTP. After 7 d of MPTP intoxication, motor tasks were assayed followed by monitoring the level of DA in the striatum. FTI and GGTI, alone or in combination, were capable of improving horizontal activity (FIG. 9B), total distance traveled (FIG. 9C), number of movements (FIG. 9D), and stereotypy time (FIG. 9E), and reversing the loss of DA (FIG. 9A) significantly in MPTP-intoxicated mice suggesting that suppression of either p21^{ras} farnesylation or p21^{rac} geranylgeranylation alone is sufficient for the protection of the nigrostriatum in MPTP-intoxicated mice.

### Example 15

### Does NaPB Halt Disease Progression in a Chronic MPTP Mouse Model?

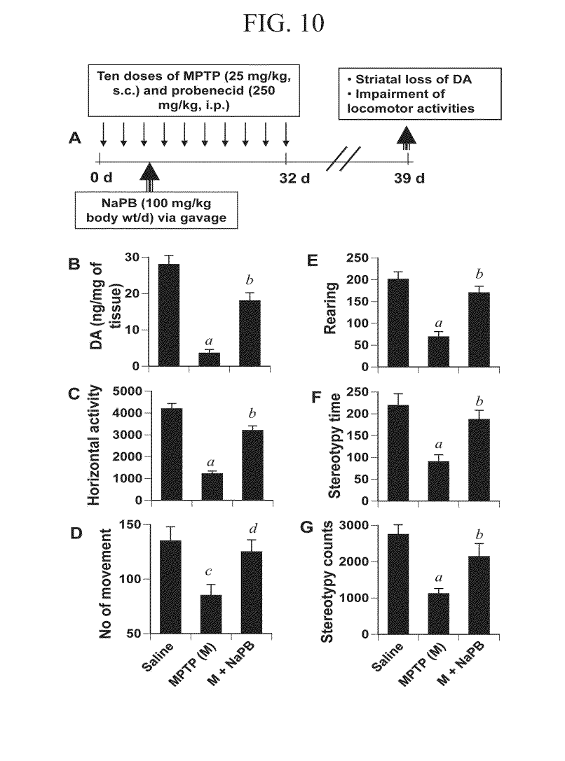

While the acute MPTP model is helpful for quick drug screening, elucidating molecular mechanisms and determining the interaction between drug and MPTP, effects of acute intoxication reverse over time. Significant loss of dopaminergic neurons is seen even six months after MPTP intoxication in a chronic intoxicated model. We examined if NaPB was capable of protecting neurons in a chronic model (FIG. 10A). As expected, chronic MPTP intoxication led to marked loss of striatal dopamine (FIG. 10B) and decrease in horizontal activity (FIG. 10C), number of movements (FIG. 10D), rearing (FIG. 10E), and stereotypy (FIG. 10E-G). We initiated oral NaPB treatment (100 mg/kg body wt/d) from the third injection of MPTP/probenecid (FIG. 10A). Consistent to that observed in the acute model (FIG. 8), NaPB, in this instance, also significantly protected striatal DA (FIG. 10B and improved locomotor activities (FIG. 10C-G).

### Example 16

### Materials and Methods Used in Examples 1-15

Animal maintaining and experiments were in accordance with National Institute of Health guidelines and were approved by the institutional Animal Care and Use committee (IACUC#06-048) of the Rush University of Medical Center, Chicago, Ill.

Isolation of Mouse Microglia: Microglia were isolated from mixed glial cultures. Briefly, on day 7-9, the mixed glial cultures were washed three times with Dulbecco's modified Eagle's medium/F-12 and subjected to shaking at 240 rpm for 2 h at 37° C. on a rotary shaker. The floating cells were washed and seeded on to plastic tissue culture flasks and incubated at 37° C. for 1 h. The attached cells were seeded onto new plates for further studies. Ninety to ninety-five percent of cells were found to be positive for Mac-1. Mouse BV-2 microglial cells (kind gift from Virginia Bocchini of Universityof Perugia) were also maintained and induced as indicated above.

Isolation of Primary Human Astroglia: Primary human astroglia were prepared. Fetal brain tissues were obtained from the Human Embryology Laboratory (University of Washington, Seattle, Wash., USA). Briefly, 11- to 17-week-old fetal brains were dissociated by trituration and trypsinization. On 9th day, these mixed glial cultures were placed on a rotary shaker at 240 rpm at 37° C. for 2 h to remove loosely attached microglia. Then on 11th day, flasks were shaken again at 190 rpm at 37° C. for 18 h to remove oligodendroglia. The attached cells remaining were primarily astrocytes. These cells were trypsinized and subcultured to yield more viable and healthy cells. More than 98% of these cells obtained by this method were positive for glial fibrillary acidic protein (GFAP), an astrocyte marker.

Immunoblot Analysis: Immunoblot analysis was carried out. Briefly, cells homogenates were electrophoresed, proteins were transferred onto a nitrocellulose membrane, and protein band was visualized with Odyssey infrared scanner after immunolabeling with primary antibodies followed by infra-red fluorophore-tagged secondary antibody (Invitrogen, Carlsbad, Calif.).

Electrophoretic Mobility Shift Assay (EMSA): Nuclear extracts were prepared and EMSA was carried out. Briefly, IRDye™ infrared dye end-labeled oligonucleotides containing the consensus binding sequence for NF-κB (5′-AGT TGA GGG GAC TTT CCC AGG C-3′ (SEQ ID NO:1)) were purchased from LI-COR Biosciences (Lincoln, Nebr.). Six-micrograms of nuclear extract was incubated with binding buffer and with IR-labeled probe for 20 min. Subsequently, samples were separated on a 6% polyacrylamide gel in 0.25× TBE buffer (Tris borate-EDTA) and analyzed by Odyssey Infrared Imaging System (LI-COR Biosciences).

Transcriptional activity of NF-κB: Transcriptional activity of NF-κB was assayed. Briefly, cells plated at 50 to 60% confluence in 12-well plates were transfected with 0.25 μg pBIIX-Luc (an NF-κB-dependent reporter construct) and 12.5 ng pRL-TK (a plasmid encodig Renilla luciferase, used as transfection efficiency control; Promega, Madison, Wis.) using LipofectAMINE Plus (Invitrogen, Carlsbad, Calif.). After 24 h of transfection, cells were stimulated with MPP+ for an additional 6 h, and firefly and Renilla luciferase activities were recorded in a TD-20/20 Luminometer (Turner Designs, Sunnyvale, Calif.) by analyzing total cell extract according to standard instructions provided in the Dual Luciferase Kit (Promega), Relative luciferase activity of cell extracts was typically represented as (firefly luciferase value/Renilta luciferase value)×10⁻³.

Chromatin immunoprecipitation (CUP): ChIP assays were performed using a ChIP assay kit (Upstate Biotechnology, Lake Placid, N.Y.). Briefly, 2×106 microglial cells preincubated with gemfibrozil for 6 h were stimulated with LPS. After 3 h of stimulation, cells were fixed by adding formaldehyde (1% final concentration), and cross-linked adducts were resuspended and sonicated, resulting in an average chromatin fragment size of 400 bp. ChIP was performed on the cell lysate by overnight incubation at 4° C. with 2 μg of antibodies against p65 followed by incubation with protein G agarose (Santa Cruz Biotechnology) for 2 h. The beads were washed and incubated with elution buMr. To reverse the cross-linking and purify the DNA, precipitates were incubated in a 65° C. incubator overnight and digested with proteinase K. DNA samples were then purified, precipitated, and precipitates were washed with 75% ethanol, air-dried, and resuspended in TE buffer. Following primers were used to amplify fragments flanking proximal NF-κB elements in the mouse iNOS promoter:

| (SEQ ID NO: 2) | |
|---|---|
| Sense: | 5′-CAT GAG GAT ACA CCA CAG AG-3′ |
| (SEQ ID NO: 3) | |
| Antisense: | 5′-AAG ACC CAA GCG TGA GGA GC-3′ |

Following primers were used to amplify fragments flanking distal NF-κB elements in the mouse iNOS promoter:

| (SEQ ID NO: 4) | |
|---|---|
| Sense: | TGC TAG GGG GAT TTT CCC TCT CTC-3′ |
| (SEQ ID NO: 5) | |
| Antisense: | 5′-ACC CTG TTC TGA GAA ACA AA-3′ |
| (SEQ ID NO: 6) | |
| Sense: | 5′-GAT GTG CTA GGG GGA TTT TCC C-3′ |
| (SEQ ID NO: 7) | |
| Antisense: | 5′-TGG GCT AGC CTG GTC TAC AGA G-3′ |

The PCRs were repeated by using varying cycle numbers and different amounts of templates in order to ensure that results were in the linear range of PCR.

Monitoring microglial ROS production: Cells, cultured in 8-well chamber slides, were treated with MPP+ under serum-free condition. At different time points of stimulation, supernatants were removed and cells were washed with Hank's buffered salt solution (HBSS) followed by addition of 100 μl of 25 μM carboxy-H2DCFDA to each well for 30 min of incubation. During the last five minutes of incubation, Hoechst 33342 was added to each well at a dilution of 1:1000 for staining nuclei. Cells were then washed with HBSS, mounted with DPX mounting media and observed under an Olympus 1×81 fluorescent microscope.

Superoxide measurements: Superoxide production was detected by LumiMax™ Superoxide Anion Detection Kit (Stratagene) as described by us (30).

Assay of cholesterol in serum: Total cholesterol was quantified irr serum by using an Amplex Red Cholesterol Assay kit from Invitrogen. Briefly, cholesterol was oxidized by cholesterol oxidase to yield H₂O₂, which then reacted with 10-acetyl-3,7 dihydroxyphenoxazine (Amplex Red). In the presence of horseradish peroxidase (HRP), this Amplex Red:H₂O₂ complex produced highly fluorescent resorufin, which was detected by fluorometry.

Animals and MPTP intoxications: Six- to eight-week old C57BL/6 mice were purchased from Harlan, Indianapolis, Ind. For acute MPTP intoxication, mice received four intraperitoneal (i.p.) injections of MPTP-HCl (18 mg/kg of free base; Sigma Chemical Co., St. Louis, Mo.) in saline at 2 hr intervals. Control animals received only saline. For chronic MPTP intoxication, mice received 10 injections of MPTP (s.c.; 25 mg/kg body weight) together with 10 injections of probenecid (i.p.; 250 mg/kg body weight) at an interval of 3.5 d.

Drugs and antibodies: NaPB, farnesyl transferase inhibitor (FTI), geranylgeranyl transferase inhibitor (GGTI), and rabbit anti-mouse iNOS were obtained from Calbiochem, Ciibbstown, N.J. Rabbit and goat anti-NF-κB p65 and goat anti-glial fibrillary acidic protein (GFAP) were purchased from Santa Cruz Biotechnology (Santa. Cruz, Calif.). Rat anti-mouse CD11b and mouse anti-human CD11b were purchased from Abeam (Cambridge, Mass.) and Serotec (Raleigh, N.C.), respectively. Cy2- and Cy5-conjugated antibodies were obtained from Jackson Immuno Research Laboratories (West Grove, Pa.).

Drug treatments: NaPB is a FDA-approved drug for patients with urea cycle disorders and its recommended dose for affected children is 400-600 mg/kg/day. However, because either PD patients or MPTP-intoxicated mice do not suffer from urea cycle disorders, we have reduced the dose for treating mice. Therefore, for short-term treatment, mice with acute MPTP 3 h after the last injection of MPTP. The neurotoxic effect of MPTP depends on several key toxicokinetic steps such as its conversion into MP+ in glial cells by MAO-B and the uptake of MPP+ by dopaminergic neurons. A sufficient amount of MPTP is converted into MPP+ within 90 min of the last injection of MPTP in an acute MPTP model. Therefore, to avoid any possible influence of NaPB on entry and conversion of MPTP into MPP+ in the midbrain, oral treatment began 3 h after the last injection of MPTP. On the other hand, for long-term treatment, mice with chronic MPTP intoxication received a lower dose of NaPB (100 mg/kg body wt/d) via gavage from the 3rd injection of MPTP/probenecid. Control MPTP mice received only 100 μl water via gavage everyday.

On the other hand, FTI and GGTI were solubilized in normal saline and mice were treated with FTI and GGTI daily via intraperitoneal (i.p.) injection at doses of 5 or 10 mg/kg body wt/d starting from 3 h after the last injection of MPTP.

Activation of p21^{ras} and p21^{rac}: Activation of p21^{ras} was monitored. Briefly, after 6 h of MPTP insult, ventral midbrain was dissected out and frozen immediately on dry ice. The p21^{ras}-binding domain (RBD) of the p21^{ras} effector kinase Rafl has been shown to bind specifically to the GTP-bound (active) form of p21^{ras} proteins. Therefore, using an assay kit from Upstate Biotechnology (Waltham, Mass.), ventral midbrain tissues were homogenized with lysis buffer containing inhibitors of different proteases and kinases followed by immuno-pull down of active p21^{ras} using Raf-RBD-GST beads. Then the amount of activated p21^{ras} was determined in GST beads by a Western blot using a p21^{ras} specific antibody.

As activated p21^{ras} interacts with Rafl, activated p21^{rac} interacts with p21-activated kinase (PAK). Accordingly, p21^{rac}-interacting domain (RID) of PAK binds specifically to the GTP-bound (active) form of p21^{rac}. Therefore, using an assay kit from Upstate Biotechnology (Waltham, Mass.), PAK-RID-GST beads were used to immuno-pull down active p21^{rac} from cell lysates followed by a Western blot using a p21^{rac} specific antibody.

Semi-quantitative RT-PCR analysis for proinflammatory molecules (iNOS, IL-1β and TNF-α) and glial cell markers: Total RNA was isolated from ventral midbrain using Ultraspec-II RNA reagent (Biotecx .Laboratories, Inc., Houston, Tex.) following manufacturer's protocol. To remove any contaminating genomic DNA, total RNA was digested with DNase. RT-PCR was carried out using a RT-PCR kit (Clontech., Mountain View, Calif.) and following primers.

iNOS:
(SEQ ID NO: 8)

| | |
|---|---|
| Sense: | 5′-CCCTTCCGAAGTTTCTGGCAGCAGC-3′ |

(SEQ ID NO: 9)

| | |
|---|---|
| Antisense: | 5′-GGCTGTCAGAGCCTCGTGGCTTTGG-3′ |

IL-1β:
(SEQ ID NO: 10)

| | |
|---|---|
| Sense: | 5′-CTCCATGAGCTTTGTACAAGG-3′ |

(SEQ ID NO: 11)

| | |
|---|---|
| Antisense: | 5′-TGCTGATGTACCAGTTGGGG-3′ |

TNF-α:
(SEQ ID NO: 12)

| | |
|---|---|
| Sense: | 5′-TTCTGTCTACTGAACTTCGGGGTGATCGGTCC-3′ |

(SEQ ID NO: 13)

| | |
|---|---|
| Antisense: | 5′-GTATGAGATAGCAAATCGGCTGACGGTGTGGG-3′ |

CD11b:
(SEQ ID NO: 14)

| | |
|---|---|
| Sense: | 5′-GTGAGGATTCCTACGGGACCCAGGT-3′ |

(SEQ ID NO: 15)

| | |
|---|---|
| Antisense: | 5′-GGCGTACTTCACAGGCAGCTCCAAC-3′ |

GFAP:
(SEQ ID NO: 16)

| | |
|---|---|
| Sense: | 5′-GGCGCTCAATGCTGGCTTCA-3′ |

(SEQ ID NO: 17)

| | |
|---|---|
| Antisense: | 5′-TCTGCCTCCAGCCTCAGGTT-3′ |

GAPDH:
(SEQ ID NO: 18)

| | |
|---|---|
| Sense: | 5′-GGTGAAGGTCGGTGTGAACG-3′ |

(SEQ ID NO: 19)

| | |
|---|---|
| Antisense: | 5′-TTGGCTCCACCCTTCAAGTG-3′ |

Real-time PCR analysis: It was performed in the ABI-Prism7700 sequence detection system (Applied Biosystems, Foster City, Calif.) using TaqMan Universal Master mix and optimized concentrations of FAM-labeled probes and primers. Data were processed by the ABI Sequence Detection System 1.6 software.

Immunohistochemistry and quantitative morphology: Seven days after MPTP intoxication, mice were sacrificed and their brains fixed, embedded, and processed for tyrosine hydroxylase (TH) staining. Total numbers of TH-positive neurons in SN pc were counted stereologically with STEREO INVESTIGATOR software (MicroBrightfield, Williston, Vt.) by using an optical fractionator. Quantitation of striatal TH immunostaining was performed. Optical density measurements were obtained by digital image analysis (Scion, Frederick, Md.). Striatal TH optical density reflected dopaminergic fiber innervation. For immunofluorescence staining on fresh frozen sections, rat anti-mouse CD11b (1:100), goat anti-mouse GFAP (1:100), rabbit anti NF-κB p65 (1:100), goat anti-NF-κB p65 (1:100), rabbit anti NF-κB p50 (1:100), and rabbit anti-mouse iNOS (1:250) were used. The samples were mounted and observed under a Bio-Rad MRC1024ES confocal laser scanning microscope.

HPLC analysis of striatal dopamine and its metabolite levels: Striatal level of dopamine, DOPAC (3,4-dihydroxyphenylacetic acid) and HVA (homovanillic acid) was quantified. Briefly, mice were sacrificed by cervical dislocation after 7 days of MPTP intoxication and their striata were collected and immediately frozen in dry ice and stored at −80° C. until analysis. On the day of the analysis, tissues were sonicated in 0.2M perchioric acid. containing isoproterenol and resulting homogenates were centrifuged at 20,000×g for 15 min at 4 C. After pH adjustment and filtration, lOul of supernatant was injected onto an Eicompak SC-3ODS column (Complete Stand-Alone HPLC-ECD System EiCOMITTEC-500 from JM Science Inc., Grand Island, N.Y.) and analyzed following manufacturer's protocol.

Analysis of GSH: Nigral tissues were dissected and then sonicated in 0.2M perchloric acid solution followed by centrifugation of nigral extracts at 12,000 rpm for 10 mins at 4° C. Resulting supernatants were analyzed for GSH in Complete Stand-Alone HPLC-ECD System EiCOMHTEC-500 using gold working electrode (Eicorn We-AU) and mobile phase containing 99% 0.1M sodium phosphate buffer (pH 2.5), 1% MeOH. (v/v), and 50 mg/L EDTA-2Na.

Behavioral analyses: Two types of behavioral experiments were conducted. This included open field experiment for locomotor activity and rotorod experiment for feet movement. .Locomotor activity was measured after 7 d of the last dose of MPTP injection in Digiscan Monitor (Omnitech Electronics, Inc., Columbus, Ohio). This Digiscan Monitor records stereotypy and rearing, behaviors that are directly controlled by striatum, as well as other basic locomotion parameters, such as horizontal activity, total distance traveled, number of movements, movement time, rest time, mean distance, mean time, center time etc. Before any insult or treatment, mice were placed inside the Digiscan Infra-red Activity Monitor for 10 min daily and on rotorod for 10 min daily for 3 consecutive days to train them and record their baseline values. Briefly, animals were removed directly from their cages and gently placed nose first into a specified corner of the open-field apparatus and after release, data acquisition began at every 5 min interval. DIGISCAN software was used to analyze and store horizontal and vertical activity data, which were monitored automatically by infra-red beams. In rotorod, the feet movement of the mice was observed at different speeds. To eliminate stress and fatigues, mice were given a 5-mM rest interval. Then 7 d after the last dose of MPTP, open field and rotorod tests were carried out twice at oh interval on each mouse separately. Locomotor activity measures were assessed after baseline value comparison.

Statistics: All values are expressed as means If: SEM. Differences among means were analyzed by one- or two-way ANOVA considering time, dose or treatment as the independent factor. The one way ANOVA was performed while analyzing dose-dependent effect of NaPB on the induction of NO production or the activation of NF-κB in activated microglial cells. On the other hand, two-way ANOVA was employed to analyze the effect of Δp21^{rac} or Δp21^{ras} on LPS-i nduced time-dependent production of superoxide. In other cases, Student's t-test was used to compare outcome between two groups (e.g. control vs MPTP, MPTP vs NaPB etc).

We claim:

1. A method of treating a neurodegenerative disorder in a subject, comprising administering an inhibitor of small G-protein activation to a subject in need thereof, wherein the small G-protein is selected from the group consisting of p21^{rac}, p21^{ras}, and the combination thereof.

2. The method of claim 1, wherein the inhibitor is selected from the group consisting of sodium phenylbutyrate (NaPB), geranylgeranyl transferase inhibitor (GGTI), farnesyl transferase inhibitor (FTI), and combinations thereof.

3. The method of claim 1, wherein the p21^{rac} is microglial p21^{rac}.

4. The method of claim 1, wherein the p21^{ras} is microglial p21^{ras}.

5. The method of claim 1, wherein the p21^{rac} is substantia nigral p21^{rac}.

6. The method of claim 1, wherein the p21^{ras} is substantia nigral p21^{ras}.

7. The method of claim 1, wherein the neurodegenerative disorder is selected from the group consisting of Parkinson's disease, Alzheimer's disease, Schizophrenia, myasthenia gravis, multiple sclerosis, microbial infections, head trauma and stroke, Pick's disease, dementia with Lewy bodies, Fiuntington disease, chromosome 13 dementias, Down's syndrome, cerebrovascular disease, Rasmussen's encephalitis, viral meningitis, NPSLE, amyotrophic lateral sclerosis, Creutzfeldt-Jacob disease, Gerstrnann-Straussler-Scheinker disease, transmissible spongiform encephalopathies, ischemic reperfusion damage (e.g. stroke), brain trauma, microbial infection, chronic fatigue syndrome, Mild Cognitive Impairment; and movement disorders (including ataxia, cerebral palsy, choreoathetosis, dystonia, Tourette's syndrome, kernicterus), tremor disorders, leukodystrophies (including adrenoleukodystrophy, metachromatic leukodystrophy, Canavan disease, Alexander disease, Pelizaeus-Merzbacher disease), neuronal ceroid lipofucsinoses, ataxia telangectasia, and Rett Syndrome.

8. The method of claim 2, wherein the NaPB inhibits p21^{rac} and p21^{ras} activation.

9. The method of claim 2, wherein the GGTI inhibits p21^{rac} activation.

10. The method of claim 2, wherein the FTI inhibits p21^{ras} activation.

11. The method of claim 9, wherein the GGTI is selected from the group consisting of GGTI-298, GGTI-2154, GGTI-2166, GGTI-286, GGTI-2166, and GGTI-DU45

12. The method of claim 10, wherein the FTI is selected from the group consisting of SCH6636, R115777, Tipifarnib (6-[amino(4-chlorophenyl)(1-methyl-1H-imidazol-5-yl)methyl]-4-(3-chlorophenyl)-1-methylquinolin-2(1H)-one), and Lonafarnib (4-(2-(4-(8-Chloro-3,10-dibromo-6,11-dihydro-5H-benzo(5,6)cyc lohepta(1,2-b)pyridin-11-yl)-1-piperidinyl)-2-oxoethyl)-1-piperidinecarboxamide).

13. The method of claim 1, wherein the inhibitor is administered orally.

14. The method of claim 1, wherein the inhibitor is administered intravenously.

15. The method of claim 1, wherein the subject is at risk of developing a neurological disorder.

16. The method of claim 1, wherein the subject is diagnosed with a neurological disorder prior to performing the method.

17. The method of claim 9, wherein the GGTI directly inhibits geranylgeranyltransferase.

18. The method of claim 10, wherein the FTI directly inhibits farnesyl transferase.

19. A method of treating a neurodegenerative disorder in a subject, comprising administering an inhibitor of farnesyl transferase to a subject in need thereof.

20. A method of treating a neurodegenerative disorder in a subject, comprising administering an inhibitor of geranylgeranyl transferase to a subject in need thereof.


SEQUENCE LISTING (19 sequences)

SEQ ID NO: 1 - DNA, 22 bp, Artificial Sequence
  Consensus binding sequence for NF-KB
agttgaggggactttcccaggc

SEQ ID NO: 2 - DNA, 20 bp, Artificial Sequence
  Synthetic primer
catgaggatacaccacagag

SEQ ID NO: 3 - DNA, 20 bp, Artificial Sequence
  Synthetic primer
aagacccaagcgtgaggagc

SEQ ID NO: 4 - DNA, 24 bp, Artificial Sequence
  Synthetic primer
tgctagggggattttccctctctc

SEQ ID NO: 5 - DNA, 20 bp, Artificial Sequence
  Synthetic primer
accctgttctgagaaacaaa

SEQ ID NO: 6 - DNA, 22 bp, Artificial Sequence
  Synthetic primer
gatgtgctagggggattttccc

SEQ ID NO: 7 - DNA, 22 bp, Artificial Sequence
  Synthetic primer
tgggctagcctggtctacagag

SEQ ID NO: 8 - DNA, 25 bp, Artificial Sequence
  Synthetic primer
cccttccgaagtttctggcagcagc

SEQ ID NO: 9 - DNA, 25 bp, Artificial Sequence
  Synthetic primer
ggctgtcagagcctcgtggctttgg

SEQ ID NO: 10 - DNA, 21 bp, Artificial Sequence
  Synthetic primer
ctccatgagctttgtacaagg

SEQ ID NO: 11 - DNA, 20 bp, Artificial Sequence
  Synthetic primer
tgctgatgtaccagttgggg

SEQ ID NO: 12 - DNA, 32 bp, Artificial Sequence
  Synthetic primer
ttctgtctactgaacttcggggtgatcggtcc

SEQ ID NO: 13 - DNA, 32 bp, Artificial Sequence
  Synthetic primer
gtatgagatagcaaatcggctgacggtgtggg

SEQ ID NO: 14 - DNA, 25 bp, Artificial Sequence
  Synthetic primer
gtgaggattcctacgggacccaggt

SEQ ID NO: 15 - DNA, 25 bp, Artificial Sequence
  Synthetic primer
ggcgtacttcacaggcagctccaac

SEQ ID NO: 16 - DNA, 20 bp, Artificial Sequence
  Synthetic primer
ggcgctcaatgctggcttca

SEQ ID NO: 17 - DNA, 20 bp, Artificial Sequence
  Synthetic primer
tctgcctccagcctcaggtt

SEQ ID NO: 18 - DNA, 20 bp, Artificial Sequence
  Synthetic primer
ggtgaaggtcggtgtgaacg

SEQ ID NO: 19 - DNA, 20 bp, Artificial Sequence
  Synthetic primer
ttggctccacccttcaagtg